(19) United States
(12) Patent Application Publication
(10) Pub. No.: US 2021/0361907 A1
(43) Pub. Date: Nov. 25, 2021

(54) MEDICAL DEVICE CENTRIC COMMUNICATION SYSTEM

(71) Applicant: SECURE SURGICAL INC, Ashville, NC (US)

(72) Inventor: David J. Hetzel, Asheville, NC (US)

(21) Appl. No.: 17/326,596

(22) Filed: May 21, 2021

Related U.S. Application Data
(60) Provisional application No. 63/028,641, filed on May 22, 2020.

(51) Int. Cl. A61M 25/00

(52) U.S. Cl. CPC A61M 25/0017; A61M 2205/3553; A61M 2205/609; A61M 2205/583; A61M 2205/3507

(57) ABSTRACT

Provided herein is a medical device centric communication system DCCS. In particular, provided herein is a communication device (e.g., for use as part of an indwelling or attached medical device) that provides secure wireless data access, storage, and quick transmission of a relevant compendium of medical device details, specific patient associated implantation images, clinical patient, and disease treatment information. The DCCS provides quick secure access to this unique compendium anytime, anywhere, regardless of patient location—to reduce unknowns, lessen liabilities, improve patient safety, reduce physician uncertainty, as well as to improve and expedite patient care.

### FIELD

Provided herein is a medical device communication system. In particular, provided herein is a communication device (e.g., for use as part of an indwelling or other medical device) that provides secure wireless data access, storage and transmission of specific patient relevance factors, medical device specifics, and implanted image information.

### BACKGROUND

Indwelling medical devices, such as indwelling catheters, are used in a variety of applications. Examples include dialysis catheters, chemotherapy ports, and devices used for delivery of other therapies.

Presently, documentation of patient medical device specifics, time and accuracy of specific device application or implantation, and the associated confirming medical images are only documented in a local medical record or local electronic medical record (EMR). In addition, images formatted to the standard Digital Imaging and Communication in Medicine (DICOM images) are available only to local/regional care providers on their local computer network dedicated to EMR and to Picture Archiving and Communication System (PACS). These electronic systems are available only to local care providers with access to these networks; they must have accepted passcodes, and specific proper ID numbers for patients and themselves. Even locally, these networks are cumbersome. One must find a network computer, log-in and input several numbers to access specific records, query and then sort to find specific medical application images then separately look to EMR procedure or surgical records to hopefully find medical device specifics, which may/may not be included.

Modern patients increasingly move in space and time from the place/time of original device acquisition and implantation. Many patients complete cancer treatments in two different regions or move about to visit family, or go on vacations. Not uncommonly, situations arise where urgent and sometimes emergent information must be obtained, but often can't be obtained from the existing system in a useful time or in a practical manner.

For example, indwelling venous catheters cannot be seen and therefore cannot be identified easily. Some catheters allow a rapid infusion pump, these are referred to as power infusion ports and some catheters do not comply with this. Patients receiving a CT scan with IV contrast need a rapid infusion pump and therefore it is important to know if the IV port is a power infusion port or not.

In addition, some patients need to have an MRI. MRIs have extremely strong magnetic force and cannot have a port or device that would be attracted by the magnet. Some indwelling catheters are MRI safe and some are not.

When a patient is receiving chemotherapy or contrast dye it is important to know that the device was applied or implanted correctly, and the catheter tip is in the inferior vena cava and not going up the neck into the internal jugular vein or across the body into the axillary vein. Without access to the medical images from the time of device placement, regional outsider radiologists or physicians cannot be sure, which introduces a medical liability. The presently available systems allow timely access only to local care providers that can connect to their EMR and PACS, but is virtually inaccessible to outsiders.

When patients leave their primary place of residence, or if they live in two separate locations, vacation, or visit children, they are now distant from their information. Intermediaries such as treating physicians, family doctors, hospitals and health systems are all unknowns to outside providers. When or if patients require care outside their original area, there are many barriers, many unknown intermediaries and many deterrents to accessing information, images, and data quickly. Access to any information is always difficult and often untimely.

What is needed are improved systems and methods for privately and securely storing and quickly accessing and distributing information, use data, and medical images regarding indwelling medical devices and their associated patient relevance factors.

### SUMMARY

Provided herein is a medical device communication system. In particular, provided herein is a communication device (e.g., for use as part of an indwelling or other medical device) that provides secure wireless data access, storage and transmission of specific patient relevance factors, medical device specifics, and implanted image information.

In contrast to existing systems, the compositions, systems, and methods described herein provide a device centric system that provides quick and simple secure access to a compendium of use, patient, device information and device application (e.g., post implantation in vivo) images. The compositions and methods of the present disclosure allow access to information wherever and whenever the device and the patient present themselves; especially when safety, medical urgency, and quick access are paramount.

Accordingly, provided herein is a composition, comprising: a medical device (e.g., indwelling medical device) comprising a computer readable medium comprising a) a wireless communications protocol and one or more of a) information about the device; b) images of the device; and c) a device specific identification number that pairs with a remote server. In some embodiments, the computer readable medium is affixed to the interior or the exterior of the medical device.

The present disclosure is not limited to particular information. Examples include, but not limited to, device ID, device age, device brand and/or model, device class and use/limitation parameters, patient information (e.g., one or more of diagnosis information and treatment information) model. In some embodiments, the images comprise images of the device in place (e.g., implanted) in a subject.

The present disclosure is not limited to particular medical devices. Examples include, but are not limited to, an indwelling IV catheter, indwelling dialysis catheter, or a surgical wire location and management device.

In some embodiments, the computer readable medium comprises a communication component selected from, for example, a radio frequency identification chip, a near field communication chip, a blue tooth communication chip, or a WiFi communication chip. In some embodiments, the information is stored on the computer readable medium or on a remote server. In some embodiments, the computer readable medium is integrated into an external or internal portion of the indwelling medical device. In some embodiments, the information is stored in a read-only format.

Further embodiments provide a system, comprising: a) a device as described herein; and b) a personal electronic device configured to access information stored on the indwelling medical device.

The present disclosure is not limited to particular personal devices. Examples include, but are not limited to, a smart phone, smart watch, tablet, or laptop computer.

Additional embodiments include a method of obtaining information, comprising: a) establishing a communication link between a device as described herein and a personal electronic device; and b) accessing information stored on the medical device with the personal electronic device using the wireless communication protocol. In some embodiments, the information is used to determine a medical course of action (e.g., performing an MRI, use of a rapid infusion port, or delivery of chemotherapy or contrast dye). In some embodiments, the method further comprises providing a patient specific passcode to the personal electronic device. In some embodiments, the passcode is required for access to the information.

Yet other embodiments provide a method of storing and retrieving medical device information, comprising: a) storing information on an indwelling medical device comprising a computer readable medium comprising a wireless communications protocol, wherein the information is selected from, for example, patient information; information about the device; images of the device; or a device specific identification number that pairs with a remote server; b) providing the indwelling medical device to a subject; and c) establishing a communication link between the indwelling medical device and a personal electronic device to access information stored on the medical device using the wireless communication protocol.

Still further embodiments provide a method of providing medical care or treatment to a subject, comprising: a) storing information on an indwelling medical device comprising a computer readable medium comprising a wireless communications protocol, wherein the information is selected from, for example, patient information; information about the device; images of the device; or a device specific identification number that pairs with a remote server; b) providing the indwelling medical device to the subject; c) establishing a communication link between the indwelling medical device and a personal electronic device to access information stored on the medical device using the wireless communication protocol; d) determining an appropriate treatment or medical care based on the information; and e) administering the treatment or medical care to the subject. In some embodiments, the device is implanted under the skin or inside the body of the subject. In some embodiments, the device is external to the body of the subject. In some embodiments, the contacting occurs at a different geographical location than the storing.

Additional embodiments are described herein.

### BRIEF DESCRIPTION OF THE DRAWINGS

FIG. 1 shows a flow chart of an exemplary workflow for input of specific information and images using a system of embodiments of the present disclosure.

FIG. 2 shows a flow chart of an exemplary workflow for retrieval of specific information and images using a system of embodiments of the present disclosure.

FIG. 3A-B shows an exemplary infusion catheter device comprising an add-on communication system of embodiments of the present disclosure.

FIG. 4A-D shows an exemplary infusion catheter device comprising an integrated communication system of embodiments of the present disclosure.

FIG. 5A-B shows an exemplary wire management device comprising an integrated communication system of embodiments of the present disclosure.

### DETAILED DESCRIPTION

Provided herein is a medical device communication system. In particular, provided herein is a communication device (e.g., for use as part of an indwelling or other medical device) that provides secure wireless data access, storage and transmission of specific patient relevance factors, medical device specifics, and image information.

Presently there are no solutions that quickly allow providers to access catheter and device specific ID needed to ensure quick or safe use outside their local areas. The present disclosure minimizes these deficiencies, allows quick and accurate device identification, specific medical images, device use parameters, and patient diagnosis specifics, which alleviates uncertainty, improves patient safety, and enables device utility.

The present disclosure provides a medical device comprising a computer readable medium chip that triggers and/or enables a patient approved provider's electronic device to access a compendium of device and patient relevant, specific medical images related to the device location and implantation, specific device ID, device age, class and use/limitation parameters. In some embodiments, the specific patient diagnoses and allergies are also stored. In some embodiments, information is read only and complies with all HIPPA and DICOM standards.

In some embodiments, the described devices and methods do not replace original PACS images and EMR records, but provide quick, specific, and powerful add-on systems to improve patient safety via enabling quick information, data and image access as patients travel outside the region of original device placement and clinical information.

Accordingly, provided herein is a composition, comprising: an indwelling medical device comprising a computer readable medium comprising a) a wireless communications protocol and one or more of a) information about the device; b) images of the device; and c) a device specific identification number that pairs with a remote server.

Safe use of the medical devise is obtained via access to otherwise disparate information and images now neatly and quickly accessed—a compendium provided/enabled by the device centric communication system described herein.

The present disclosure is not limited to particular information. Examples include, but not limited to, device ID, device age, device brand, device class and use parameters, patient information (e.g., one or more of facility (e.g., original facility) information, patient name, age, and date of birth, diagnosis information, patient allergies, treatment information, or other medically relevant health information), or device model and/or limitations.

The present disclosure is not limited to a particular image type or format. In some embodiments, the images comprise images of the device in place in a subject. In some embodiments, images are X-rays, CT scans, MRIs, or other imaging modalities. In some embodiments, the images are digital Imaging and Communications in Medicine (DICOM) images. DICOM is the standard for the communication and management of medical imaging information and related data. DICOM is most commonly used for storing and transmitting medical images enabling the integration of medical imaging devices such as scanners, servers, workstations, printers, network hardware, and picture archiving and communication systems (PACS) from multiple manufacturers. It has been widely adopted by hospitals and is making inroads into smaller applications like dentists' and doctors' offices.

DICOM files can be exchanged between two entities that are capable of receiving image and patient data in DICOM format. In some embodiments, the different devices come with DICOM Conformance Statements, which state which DICOM classes they support. The standard includes a file format definition and a network communications protocol that uses TCP/IP to communicate between systems.

In some embodiments, the computer readable medium comprises a communication component selected from, for example, a radiofrequency identification chip, a near field communication chip, a blue tooth communication chip, or a WiFi communication chip.

Radio-frequency identification (RFID) uses electromagnetic fields to automatically identify and track tags attached to objects. An RFID tag consists of a tiny radio transponder; a radio receiver and transmitter. When triggered by an electromagnetic interrogation pulse from a nearby RFID reader device, the tag transmits digital data, usually an identifying inventory number, back to the reader. This number can be used to inventory goods. There are two types. Passive tags are powered by energy from the RFID reader's interrogating radio waves. Active tags are powered by a battery and thus can be read at a greater range from the RFID reader; up to hundreds of meters. Unlike a barcode, the tag doesn't need to be within the line of sight of the reader, so it may be embedded in the tracked object.

Near-field communication (NFC) is a set of communication protocols by which two electronic devices communicate when they are brought within 4 cm (1½ in) of each other. NFC devices are used in contactless payment systems, similar to those used in credit cards and electronic ticket smart cards and allow mobile payment to replace or supplement these systems. This is sometimes called NFC/CTLS and CTLS NFC, with contactless abbreviated CTLS. NFC is used for social networking and for sharing contacts, photos, videos, and other files. NFC devices can act as electronic identity documents and keycards. NFC offers a low-speed connection with simple setup that can be used to bootstrap more-capable wireless connections.

The Secure Element chip is an NFC chip that contains data such as the Secure Element Identifier (SEID) for secure transactions. This chip is commonly found in smartphones and other NFC devices.

Bluetooth is a wireless technology standard used for exchanging data between fixed and mobile devices over short distances using short-wavelength UHF radio waves in the industrial, scientific and medical radio bands, from 2.400 to 2.485 GHz, and building personal area networks (PANs). It was originally conceived as a wireless alternative to RS-232 data cables.

Wi-Fi is a family of wireless networking technologies, based on the IEEE 802.11 family of standards, which are commonly used for local area networking of devices and Internet access. Wi-Fi uses multiple parts of the IEEE 802 protocol family and is designed to interwork seamlessly with its wired sibling Ethernet. Compatible devices can network through a wireless access point to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 gigahertz (120 mm) UHF and 5 gigahertz (60 mm) SHF ISM radio bands; these bands are subdivided into multiple channels. Channels can be shared between networks, but only one transmitter can locally transmit on a channel at any moment in time.

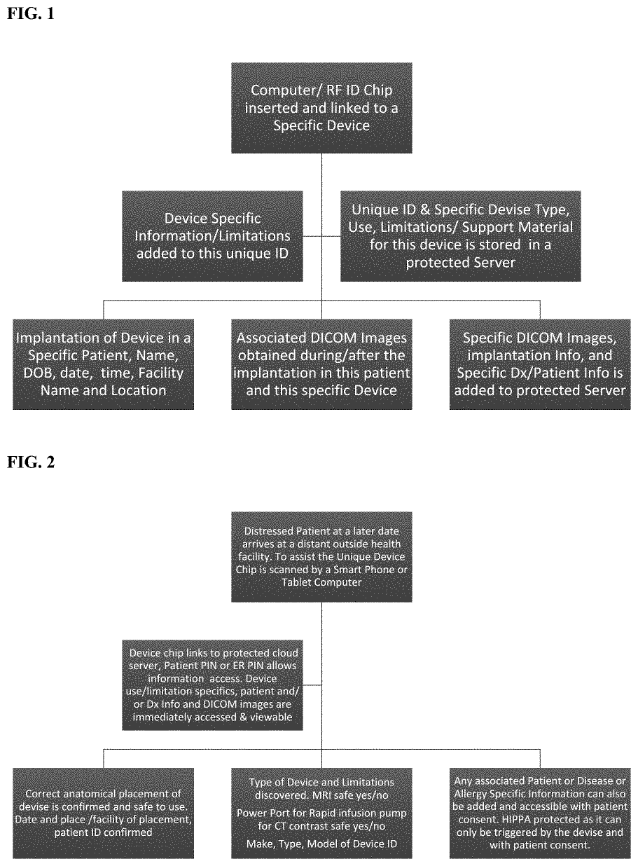

FIGS. 1-2 show exemplary workflows for device, systems, and methods of embodiments of the disclosure. FIG. 1 shows specific information and image input, device centric and device triggered patient specific storage of DICOM Images, clinical application information, and device use/limitation specifics. A RFID or other chip with a unique ID is inserted and linked to a specific device (e.g., indwelling catheter or other medical device). Next, device specific information is added to this unique ID. The unique ID and device information is stored in a protected server or on the chip. In some embodiments, these steps are conducted prior to placement of the device in or on the subject. The device is then assigned and placed in or on the subject. Information on the subject (e.g., name, date of birth, time of placement, facility name and location, etc.) are added to the device information on the server or chip. In some embodiments, additional information such as patient medications, allergies, treatment plan, etc. are included. In some embodiments, associated images (e.g., DICOM images) obtained during or after application of the device to the subject are also added to the device information on the chip or server.

FIG. 2 shows device centric and device triggered patient specific retrieval of DICOM Images, clinical application information, and device use/limitation specifics. A subject (e.g., in need of medical care or follow-up) arrives (e.g., at a different location or clinical network). The new provider scans the device (e.g., using a smart phone, tablet, or other portable device) to assess the unique device ID and associated information. The device chip provides or links to the remote server to obtain information and images. In some embodiments, a patient specific or provider specific PIN or passcode is required to access information. The new provider is then able to confirm device information and correct placement/usage of device. In some embodiments, the provider uses the information to provide a treatment course of action (e.g., to proceed with infusion or MRI or recommend replacement/modification of the device). In some embodiments, the new provider is able to add additional information (e.g., treatments provided, new patient information, etc.).

In some embodiments, the information is stored on the computer readable medium or on a remote server. In some embodiments, the commuter readable medium is integrated into an external or internal portion of the medical device. In some embodiments, the information is stored in a read-only format.

In some embodiments, the secure storage of information is on the computer readable medium or in a privacy protected computer server that is accessible wirelessly. Examples include, but are not limited to, a third-party protected cloud site, or a device manufacturer protected site. In some embodiments, as described herein, information is accessible only by patient consent, only by scanning of the patient applied device with the ID chip, and/or only with proper approved passcodes.

As described herein, in some embodiments, information stored on the medical device is accessed by personal electronic device.

The present disclosure is not limited to particular personal devices. Examples include, but are not limited to, a smart phone, smart watch, tablet, or laptop computer.

To trigger, sort, and retrieve the needed information at a later date from other places around the world, the patient allows a health care provider to a bring a tablet computer, smartphone, RF-ID Reader, laptop computer or other personal device within range (e.g., 2 inches to multiple feet depending on the communication technology of the device) so it can read the chip information by NFC, RF-ID, Blue tooth, or WiFi. The patient may also need to supply a passcode (e.g., PIN) during the process. The wireless device then allows the medical device to trigger, sort, and retrieve the above information, allowing the personal device displays the specific device documents, application DICOM images and any included medical diagnoses, or added patient information. In some embodiments, the access by wireless devices is read-only.

In some embodiments, access to information requires a patient specific passcode to the personal electronic device. In some embodiments, the passcode is required for access to the information. In some embodiments, patients pre-approve and sign for device triggered retrieval of specific diagnostic images, device specifics, and diagnoses information to outside providers in urgent/emergent times, or in cases where the patient is in need (e.g., outside their usual region of intervention / treatment).

Once viewed by a medical health care provider or radiologist, information is read, but not copied or forwarded, and not printed. In some embodiments, it is possible to add to the stored file images or medical diagnoses, and interventions, if the patient agrees. However, in some embodiments, nothing can be edited out or altered from the past device centric record. For example, in some embodiments, previous information, along with new ongoing diagnoses, data, images, can be added, triggered, accessed, and read by future caregivers at any distant sites as needs arise.

In some embodiments, the data storage, access, and communication compositions and methods described herein are in compliance with the Health Insurance Portability and Accountability Act of 1996 (HIPAA or the Kennedy-Kassebaum Act). The HIPAA Privacy Rule is composed of national regulations for the use and disclosure of Protected Health Information (PHI) in healthcare treatment, payment and operations by covered entities. The HIPAA Privacy Rule regulates the use and disclosure of protected health information (PHI) held by “covered entities” (generally, health care clearinghouses, employer-sponsored health plans, health insurers, and medical service providers that engage in certain transactions). By regulation, the HHS extended the HIPAA privacy rule to independent contractors of covered entities who fit within the definition of “business associates”. PHI is any information that is held by a covered entity regarding health status, provision of health care, or health care payment that can be linked to any individual. This is interpreted rather broadly and includes any part of an individual's medical record or payment history. Covered entities must disclose PHI to the individual within 30 days upon request. Also, they must disclose PHI when required to do so by law such as reporting suspected child abuse to state child welfare agencies.

A covered entity may disclose PHI to certain parties to facilitate treatment, payment, or health care operations without a patient's express written authorization. Any other disclosures of PHI require the covered entity to obtain written authorization from the individual for the disclosure. In any case, when a covered entity discloses any PHI, it must make a reasonable effort to disclose only the minimum necessary information required to achieve its purpose.

Under HIPAA, HIPAA-covered health plans are now required to use standardized HIPAA electronic transactions. See, 42 USC § 1320d-2 and 45 CFR Part 162. Information about this can be found in the final rule for HIPAA electronic transaction standards (74 Fed. Reg. 3296, published in the Federal Register on Jan. 16, 2009), and on the CMS website.

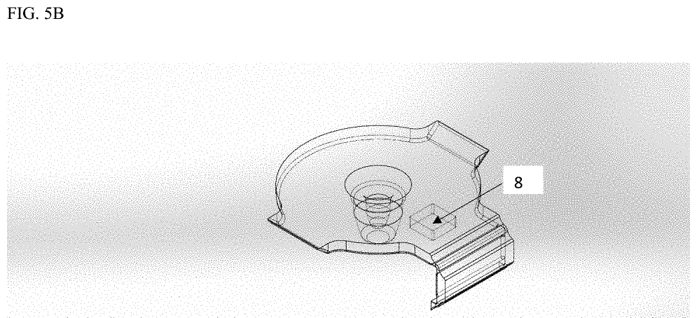

The present disclosure is not limited to particular medical devices. Examples include, but are not limited to, an indwelling IV catheter, indwelling dialysis catheter, or a surgical wire management device (e.g., described in U.S. Pat. No. 10,688,286; herein incorporated by reference in its entirety) and shown in FIGS. 5A-B. In some embodiments, the devices described herein utilize commercially available indwelling catheters.

In some embodiments, the device is an indwelling IV catheter or port. A port (e.g., available from Smiths Medical, Dublin, Ohio) is an implanted device which allows easy access to a patient's veins. A port is surgically-inserted completely beneath the skin and consists of two parts—the portal and the catheter. The portal is typically made from a silicone bubble and appears as a small bump under the skin. The portal, made of self-sealing silicone, can be punctured by a needle repeatedly before the strength of the material is compromised. Its design contributes to a very low risk of infection. The slender, plastic catheter attached to the portal is threaded into a central vein (usually the jugular vein, subclavian vein, or the superior vena cava). Ports are indicated for patients requiring frequent and long-term intravenous therapy, such as the oncology population.

A port can be single or double lumen. Single lumen ports are most common and typically sufficient for patients requiring scheduled intravenous therapy.

In some embodiments, the port is a power injection port (e.g., available from BD, Tempe Ariz). Power injection ports can withstand higher injection pressures.

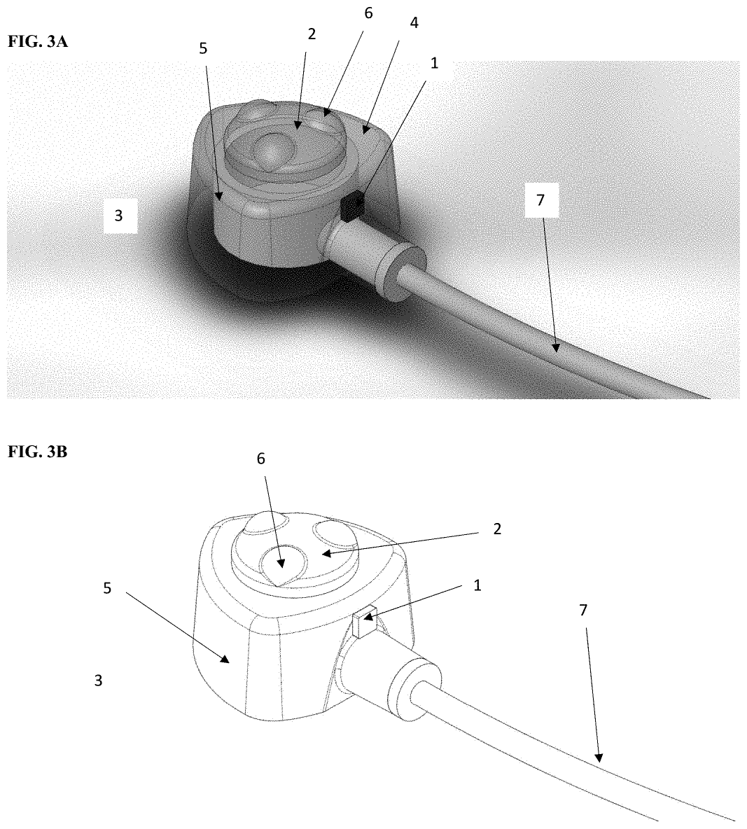

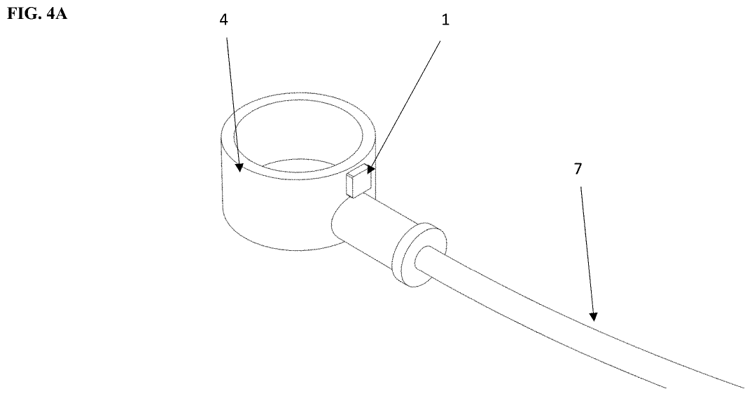

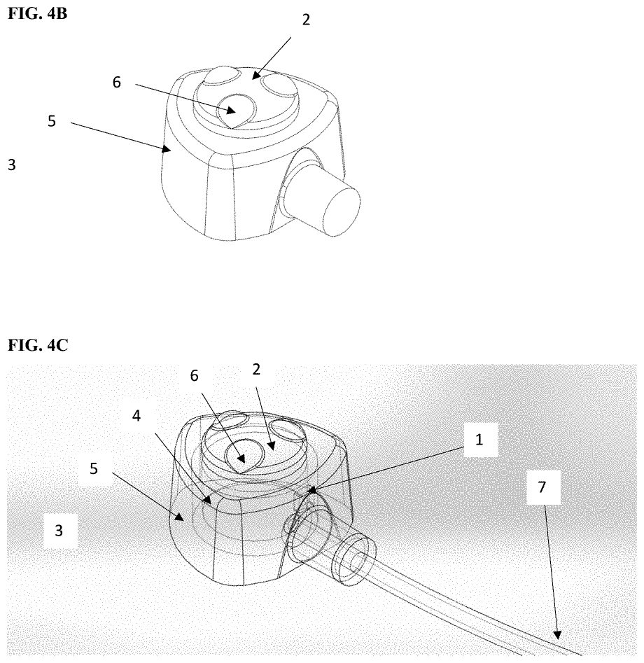

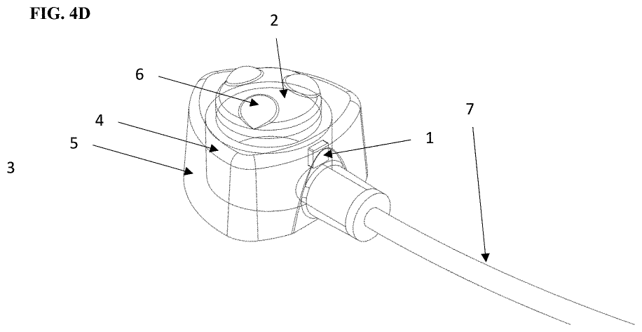

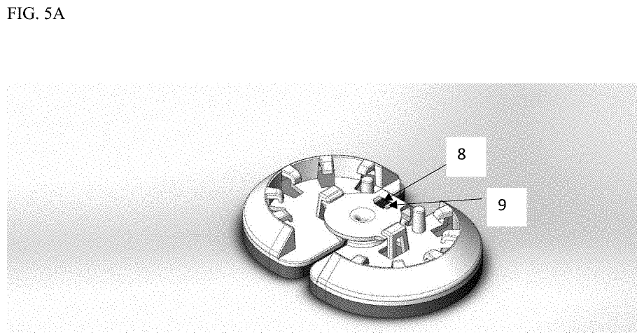

FIGS. 3-4 show exemplary power injection port devices comprising computer readable medium devices of embodiments of the present disclosure. While the figures illustrate a power injection port, the devices and systems of the present disclosure are suitable for use with any number of indwelling IV catheters and other devices.

FIG. 3 shows an exemplary power injection port with computer readable medium chip (CRM chip) on the outside of the device (e.g., added after or during manufacture of the device). FIG. 3A shows a transparent model of a power injection port with CRM chip. Shown is device 3, CRM chip 1, lumen 2, lumen support 4, outer housing 5, and location bumps 6. Also shown is tubing 7. In the embodiment shown in FIG. 3, the CRM chip is affixed to the exterior of the device using any suitable method (e.g., adhesive, molding, heat, etc.).

In order to access the port, a medical professional punctures the lumen 2 with a needle. Locations bumps 6 allow the user to locate the lumen. The treatment is then delivered through the needle, entering a vein via tubing 7.

FIG. 3B shows a translucent model of a power injection port with CRM chip. Shown is device 3, CRM chip 1, lumen 2, outer housing 5, and location bumps 6. Also shown is tubing 7. The lumen support 4 is not visible.

FIG. 4 shows an embodiment where the CRM chip is located at an interior location of the device (e.g., added during manufacture of the device). FIG. 4A shows CRM chip 1 affixed to lumen support 4. However, the present disclosure is not limited to the interior location shown in FIG. 4. For example, in some embodiments, the CRM chip is affixed adjacent to an inner surface of outer housing 5 (not shown). Also shown is tubing 7.

FIG. 4B shows a translucent model of a power injection port with CRM chip affixed adjacent to lumen support 4. Shown is device 3, lumen 2, outer housing 5, and location bumps 6. The chip 1 and lumen support 4 are not visible in the translucent image shown in FIG. 4B.

FIG. 4C shows a transparent model of a power injection port with CRM chip affixed adjacent to lumen support 4. Shown is device 3, CRM chip 1, lumen 2, lumen support 4, outer housing 5, and location bumps 6. Also shown is tubing 7.

FIG. 4D shows a semi-transparent model of a power injection port with CRM chip affixed adjacent to lumen support 4. Shown is device 3, CRM chip 1, lumen 2, lumen support 4, outer housing 5, and location bumps 6. Also shown is tubing 7.

FIGS. 5A-B shows exemplary guide wire management devices comprising a computer readable medium chip (CRM chip). FIG. 5A shows CRM chip 8 and CRM chip securing tabs 9. The CRM securing tabs 9 keep the CRM chip 8 in place.

FIG. 5B shows an embodiment of a guide wire management device where a CRM chip 8 is embedded in the device without any additional securing component. For example, in some embodiments, the CRM chip 8 is added during manufacture (e.g., molding) or a slot or hole is machined in the device after manufacture.

The present disclosure is not limited to the physical locations of computer readable medium chips on devices shown in the figures. Any suitable location on the medical device is specifically contemplated.

In some embodiments, the information is used to recommend, determine, or administer a medical course of action (e.g., performing an MRI, use of a rapid infusion port, or delivery of chemotherapy or contrast dye). In some embodiments, the treatment course of action is administered to the patient or subject.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. An indwelling medical device, comprising:
a computer readable medium comprising a) a wireless communications protocol and one or more of a) information about the device; b) images of the device; and c) a device specific identification number that pairs with a remote server.

2. A surgical wire management device, comprising:
a computer readable medium comprising a) a wireless communications protocol and one or more of a) information about the device; b) images of the device; and c) a device specific identification number that pairs with a remote server.

3. The device of claim 1, wherein said information is selected from the group consisting of device ID, device age, device brand, device class and use parameters, and device model.

4. The device of claim 1, wherein said images comprise images of the device in place in a subject in vivo and/or patient medical images.

5. The medical device of claim 1, wherein indwelling medical device is selected from the group consisting of an indwelling implanted IV catheter and an indwelling dialysis catheter

6. The medical device of claim 1, wherein said computer readable medium comprises a communication component selected from the group consisting of a radiofrequency identification chip, a near field communication chip, a blue tooth communication chip, and a WiFi communication chip.

7. The medical device of claim 1, wherein said information is stored on said computer readable medium or on said remote server.

8. The medical device of claim 1, wherein said computer readable medium is integrated into an external or internal portion of said indwelling medical device.

9. The medical device of claim 1, wherein said computer readable medium further comprises patient and/or treatment information.

10. The medical device of claim 9, wherein said patient information comprises one or more of name, date of birth, facility information, diagnosis information, treatment and/or infused drug information, allergy information, co-morbidities, and health information.

11. The medical device of claim 1, wherein said information is stored in a read-only format.

12. The medical device of claim 1, wherein said computer readable medium is affixed to the interior or the exterior of said indwelling medical device.

13. A method of retrieving medical device information, comprising:
a) establishing a communication link between the medical device of claim 1 and a personal electronic device; and
b) accessing information stored on said medical device with said personal electronic device using said wireless communication protocol.

14. The method of claim 13, wherein said information informs a user of a compendium of specific device and patient relevant information, data, and in vivo images.

15. The method of claim 13, wherein said information is used to determine a medical course of action.

16. The method of claim 15, wherein said medical course of action is selected from the group consisting of performing an MRI, use of a rapid infusion port, guiding a procedure, performing dialysis, and delivery of chemotherapy or contrast dye.

17. The method of claim 13, further comprising providing a patient specific passcode to said personal electronic device.

18. The method of claim 17, wherein said passcode is required for access to said information.

19. A method of providing medical care or treatment to a subject, comprising:
a) storing information on a medical device comprising a computer readable medium comprising a wireless communications protocol, wherein said information is selected from the group consisting of patient information; information about the device;
patient specific medical images of the device in place; and a device specific identification number that pairs with a remote server;
b) providing said medical device to said subject;
c) establishing a communication link between said medical device and a personal electronic device to access information stored on said medical device using said wireless communication protocol;
d) determining an appropriate treatment or medical care based on said information; and
e) administering said treatment or medical care to said subject.

20. The method of claim 19, wherein said device is implanted under the skin or inside the body of said subject.
